(19) United States
(12) Patent Application Publication
(10) Pub. No.: US 2026/0053939 A1
(43) Pub. Date: Feb. 26, 2026

(54) Combination Therapy for Cancer

(71) Applicant: GlaxoSmithKline Intellectual Property Development Limited, Stevenage (GB)

(72) Inventors: Geraldine FERRON-BRADY, Collegeville, PA (US); Morrys C. KAISERMANN, Collegeville, PA (US); Brandon KREMER, Collegeville, PA (US); Alessandra TOSOLINI, Collegeville, PA (US); Xiaoou Linnette ZHOU, Boston, MA (US)

(73) Assignee: GlaxoSmithKline Intellectual Property Development Limited, Stevenage (GB)

(21) Appl. No.: 18/730,820

(22) PCT Filed: Jan. 24, 2023
(86) PCT No.: PCT/IB2023/050600

§ 371 Date: Jul. 22, 2024

Related U.S. Application Data
(60) Provisional application No. 63/302,924, filed on Jan. 25, 2022.

(51) Int. Cl. A61K 47/68; A61K 31/454; A61K 31/573; A61K 31/69; A61P 35/00

(52) U.S. Cl. CPC A61K 47/68031; A61K 31/454; A61K 31/573; A61K 31/69; A61K 47/6849; A61P 35/00

(57) ABSTRACT

This disclosure provides combination therapies comprising an anti-BCMA antigen binding protein, such as belantamab mafodotin; an immunomodulatory imide drug (IMiD); a proteasome inhibitor; and a corticosteroid. This disclosure also provides combination therapies for treating newly diagnosed multiple myeloma.

### CROSS-REFERENCE TO RELATED APPLICATIONS

This application is a National Stage of International Application No. PCT/IB2023/050600, filed 24 Jan. 2023, which claims the benefit of U.S. Provisional Application No. 63/302,924, filed 25 Jan. 2022, both of which are incorporated herein by reference in their entireties.

### REFERENCE TO SEQUENCE LISTING SUBMITTED ELECTRONICALLY

The instant application contains a Sequence Listing which has been submitted in XML format via EFS-Web and is hereby incorporated by reference in its entirety. Said XML copy, created on 24 Jan. 2023, is named “Sequence Listing.xml” and is 12 KB in size.

Each reference cited in this disclosure is incorporated herein by reference in its entirety.

### TECHNICAL FIELD

This disclosure relates generally to combination therapies for newly diagnosed multiple myeloma.

### BACKGROUND

Treatment of newly diagnosed multiple myeloma (NDMM) has changed dramatically over the past 20 years, from near uniform application of chemotherapy to a patient performance status- and risk-based approach. The standard of care for fit multiple myeloma patients is to receive high-dose chemotherapy, with autologous stem cell transplant, after completion of induction therapy. However, some patients, especially the elderly, are ineligible for transplant due to frailty and/or comorbidities. For such patients, treatment with highly active doublet or triplet combination regimens such as lenalidomide (REVLIMID®)/dexamethasone (Rd); daratumumab (DARZALEX®) plus Rd; or bortezomib (VELCADE®), lenalidomide (REVLIMID®), and dexamethasone (VRd) are considered.

The choice of therapy in newly diagnosed MM (NDMM) patients depends largely on an individual's suitability for autologous stem cell transplant (ASCT). ASCT is usually only suitable for fitter patients, and typically younger than 65-75 years old; therefore, transplant ineligible (TI) patients constitute a large proportion of NDMM patients. Clinical prognosis for these patients is worse in terms of progression-free survival (PFS), overall survival (OS), or achieved minimal residual disease (MRD) negativity rates compared to patients who can undergo ASCT. Thus, there is an unmet clinical need to improve prognosis through the addition of effective novel therapies to standard of care (SoC) first-line NDMM backbone agents.

### SUMMARY

One aspect of the disclosure is a combination therapy comprising (a) an anti-B-cell maturation antigen (BCMA) antigen binding protein, such as belantamab mafodotin; (b) a therapeutically effective dose of a proteasome inhibitor, such as bortezomib; (c) a therapeutically effective dose of an immunomodulatory imide drug, such as lenalidomide; and (d) a therapeutically effective amount of a corticosteroid, such as dexamethasone.

Another aspect of the disclosure is a method of treating newly diagnosed multiple myeloma, comprising administering therapeutically effective doses of components of a combination therapy.

Another aspect of the disclosure are kits. In some embodiments, a kit disclosed herein is for use in treatment of newly diagnosed multiple myeloma. In some embodiments, a kit disclosed herein can comprise a combination disclosed herein and instructions for use in the treatment.

Yet another aspect of the disclosure are pre-filled syringes or autoinjector devices. In some embodiments, a pre-filled syringe or autoinjector device disclosed herein can comprise a combination disclosed herein.

### DETAILED DESCRIPTION

This disclosure provides combination therapies and their use for treating newly diagnosed multiple myeloma. This disclosure also provides combination therapies for maintenance treatment of multiple myeloma. “Combination therapy” as used herein means a therapy comprising two or more active agents. The two or more active agents can be administered in separate dosage forms (e.g., a first agent could be lyophilized powder, a second agent could be aqueous solution, and a third or more agents could be a tablet, capsule, or other oral dosage form). The two or more active agents can be administered at the same time or at separate times and can each be administered by the same route or by different routes (e.g., a first agent could be administered intravenously, a second agent could be administered subcutaneously, and a third or more agents could be administered orally). “Newly diagnosed multiple myeloma” as used herein means multiple myeloma not previously treated with a standard of care (SoC) treatment for multiple myeloma.

In some embodiments, the disclosed combination therapies comprise an anti-BCMA antigen binding protein, such as belantamab mafodotin, and an SoC for newly diagnosed multiple myeloma. In some embodiments, the SoC comprises an immunomodulatory imide drug and a corticosteroid. In some embodiments, the SoC comprises an immunomodulatory imide drug, an anti-CD38 antibody, and a corticosteroid. In some embodiments, the SoC comprises a proteasome inhibitor, an immunomodulatory imide drug, and a corticosteroid. In some embodiments, any of the combination therapies further comprise a gamma-secretase inhibitor.

In some embodiments, the disclosed combination therapies comprise an anti-BCMA antigen binding protein, such as belantamab mafodotin, and an SoC for maintenance treatment of multiple myeloma. In some embodiments, the SoC comprises an immunomodulatory imide drug and a corticosteroid. In some embodiments, any of the combination therapies further comprise a gamma-secretase inhibitor.

“Q3W” refers to administration once every three weeks. “Q4W” refers to administration once every four weeks. “Q6W” refers to administration once every six weeks. “Q8W” refers to administration once every eight weeks. “Q10W” refers to administration once every ten weeks. “Q12W” refers to administration once every twelve weeks.

“Q3/4W” refers to administration once every three weeks for an initial period of time followed by administration once every four weeks for a subsequent period of time. In some embodiments, “Q3/4W” refers to administration once every three weeks for eight cycles, wherein each cycle is 21 days, followed by administration once every four weeks from cycle 9 onwards. “Q6/8W” refers to administration once every six weeks for an initial period of time followed by administration once every eight weeks for a subsequent period of time. In some embodiments, “Q6/8W” refers to administration once every six weeks for eight cycles, wherein each cycle is 21 days, followed by administration once every eight weeks from cycle 9 onwards. “Q9/12W” refers to administration once every nine weeks for an initial period of time followed by administration once every twelve weeks for a subsequent period of time. In some embodiments, “Q9/12W” refers to administration once every nine weeks for eight cycles, wherein each cycle is 21 days, followed by administration once every twelve weeks from cycle 9 onwards.

### Components of Combination Therapies

### Anti-BCMA Antigen Binding Proteins

The term “anti-BCMA antigen binding protein” as used herein refers to antibodies and other protein constructs, such as domains, that are capable of binding to BCMA. The terms “BCMA binding protein” and “BCMA antigen binding protein” are used interchangeably herein. This does not include the natural cognate ligand or receptor.

The anti-BCMA antigen binding proteins described herein may bind to human BCMA including, for example, human BCMA containing the amino acid sequence of GenBank Accession Number Q02223.2, or genes encoding human BCMA having at least 90 percent homology or at least 90% identity thereto.

Examples of anti-BCMA antigen binding proteins and methods of making the same are disclosed in International Publication No. WO2012/163805. Additional exemplary anti-BCMA antigen binding proteins include those described in WO2016/014789, WO2016/090320, WO2016/090327, WO2016/020332, WO2016/079177, WO2014/122143, WO2014/122144, WO2017/021450, WO2016/014565, WO2014/068079, WO2015/166649, WO2015/158671, WO2015/052536, WO2014/140248, WO2013/072415, WO2013/072406, WO2014/089335, US2017/165373, WO2013/154760, WO2018/201051, and WO2017/051068.

In some embodiments, the anti-BCMA antigen binding protein is an anti-BCMA antibody. The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., a domain antibody (DAB)), antigen binding antibody fragments, Fab, F(ab′)₂, Fv, disulfide linked Fv, single chain Fv, disulfide-linked scFv, diabodies, Tandem diabodies (TandAbs), etc. and modified versions of any of the foregoing (for a summary of alternative “antibody” formats see Holliger and Hudson, Nature Biotechnology, 2005, Vol 23, No. 9, 1126-1136).

Full, whole, or intact antibody, used interchangeably herein, refers to a heterotetrameric glycoprotein with an approximate molecular weight of 150,000 Daltons. An intact antibody is composed of two identical heavy chains (HCs) and two identical light chains (LCs) linked by covalent disulfide bonds. This H₂L₂ structure folds to form three functional domains comprising two antigen-binding fragments, known as ‘Fab’ fragments, and a ‘Fc’ crystallizable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or λ, light chain. The majority of antibodies in the serum belong to the IgG class, there are four isotypes of human IgG (IgG1, IgG2, IgG3 and IgG4), the sequences of which differ mainly in their hinge region.

Fully human antibodies can be obtained using a variety of methods, for example using yeast-based libraries or transgenic animals (e.g., mice) that are capable of producing repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunized with an antigen of interest and antigen-specific human antibodies isolated using B-cell sorting techniques. Human antibodies produced using these techniques can then be characterized for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR,” “CDRL1,” “CDRL2,” “CDRL3,” “CDRH1,” “CDRH2,” “CDRH3” follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

In some embodiments, an anti-BCMA binding protein comprises any one or a combination of the following CDRs shown in the table below.

TABLE 1
Examples CDR sequences for an anti-BCMA
antigen binding protein.

| CDR | Sequence | SEQ ID NO: |
|---|---|---|
| V_{H} CDR1 | NYWMH | 1 |
| (CDRH1) | | |
| V_{H} CDR2 | ATYRGHSDTYYNQKFKG | 2 |
| (CDRH2) | | |
| V_{H} CDR3 | GAIYDGYDVLDN | 3 |
| (CDRH3) | | |
| V_{L} CDR1 | SASQDISNYLN | 4 |
| (CDRL1) | | |
| V_{L} CDR2 | YTSNLHS | 5 |
| (CDRL2) | | |
| V_{L} CDR3 | QQYRKLPWT | 6 |
| (CDRL3) | | |

CDRs may be modified by at least one amino acid substitution, deletion or addition, wherein the variant antigen binding protein substantially retains the biological characteristics of the unmodified protein, such as binding to the antigen.

In some embodiments, the anti-BCMA antigen binding protein comprises CDRH1 according to SEQ ID NO:1, CDRH2 according to SEQ ID NO:2, CDRH3 according to SEQ ID NO:3, CDRL1 according to SEQ ID NO:4, CDRL2 according to SEQ ID NO:5, and CDRL3 according to SEQ ID NO:6.

In some embodiments, the anti-BCMA antigen binding protein comprises CDR sequences which have at least 90% or 95% or 99% sequence identity to CDRH1 according to SEQ ID NO:1, CDRH2 according to SEQ ID NO:2, CDRH3 according to SEQ ID NO:3, CDRL1 according to SEQ ID NO:4, CDRL2 according to SEQ ID NO:5, and/or CDRL3 according to SEQ ID NO:6. “Percent identity” or “% identity” between a query amino acid sequence and a subject amino acid sequence is the “Identities” value, expressed as a percentage, that is calculated using a suitable algorithm (e.g., BLASTP, FASTA, Needleman-Wunsch, Smith-Waterman, LALIGN, or GenePAST/KERR) or software (e.g., DNASTAR® LASERGENE®, GENOMEQUEST®, EMBOSS needle or EMBOSS infoalign), over the entire length of the query sequence after a pair-wise global sequence alignment has been performed using a suitable algorithm (e.g., Needleman-Wunsch or GenePAST/KERR) or software (e.g., DNASTAR® LASERGENE® or GenePAST/KERR). Importantly, a query amino acid sequence may be described by an amino acid sequence disclosed herein, in particular in one or more of the claims.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. In the case of nucleic acid sequences, such alterations include at least one nucleotide residue deletion, substitution or insertion, wherein said alterations may occur at the 5′- or 3′-terminal positions of the query sequence or anywhere between those terminal positions, interspersed either individually among the nucleotide residues in the query sequence or in one or more contiguous groups within the query sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence.

For antibody sequences, the % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g., the framework sequence, so that the CDR sequences are fixed and intact.

In some embodiments, the anti-BCMA antigen binding protein comprises a heavy chain variable region (VH) according to SEQ ID NO:7 and a light chain variable region (VL) according to SEQ ID NO:8.

SEQ ID NO: 7
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS
SEQ ID NO: 8
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKR

In some embodiments, the anti-BCMA antigen binding protein comprises a heavy chain (H) according to SEQ ID NO:9 and a light chain (L) according to SEQ ID NO:10.

SEQ ID NO: 9
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
SEQ ID NO: 10
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, the anti-BCMA antigen binding protein is a T-cell redirecting antibody (BiTE) with dual inhibition of BCMA and CD3 receptors, such as teclistamab (Pillarisetti et al., Blood Advances 4, 4538-49, 2020) and blinatumomab, AMG 424, GBR 1342, BFR4350A, AMG 420, AMG 701, elranatamab (PF-06863135), REGN5458, TNB-383B (Alhallak et al., Cancers 13, 2853, 2021). In some embodiments, the anti-BCMA antigen binding protein is an afucosylated BCMA-directed antibody, such as SEA-BCMA (Van Epps et al., Cancer Res 2018; 78(13 Suppl):Abstract nr 3833).

### CAR T Cell Therapeutics

In some embodiments, the anti-BCMA antigen binding protein is a CAR-T cell therapeutic, such as bb2121, ALLO-715, or PBCAR269A. See Raje et al., N. Engl. J. Med. 380, 1726-37, 2019); Abramson, Int. J. Mol. Sci. 21, 5192, 2020. The term “chimeric antigen receptor” (“CAR”) as used herein, refers to an engineered receptor that consists of an extracellular antigen binding domain (usually derived from a monoclonal antibody, or fragment thereof, e.g., a VH domain and a VL domain in the form of a scFv), optionally a spacer region, a transmembrane region, and one or more intracellular effector domains. CARs have also been referred to as chimeric T cell receptors or chimeric immunoreceptors (CIRs). CARs are genetically introduced into hematopoietic cells, such as T cells, to redirect T cell specificity for a desired cell-surface antigen, resulting in a CAR-T therapeutic.

The term “spacer region” as used herein, refers to an oligo- or polypeptide that functions to link the transmembrane domain to the target binding domain. This region may also be referred to as a “hinge region” or “stalk region.” The size of the spacer can be varied depending on the position of the target epitope in order to maintain a set distance (e.g., 14 nm) upon CAR:target binding. The term “transmembrane domain” as used herein, refers to the part of the CAR molecule that traverses the cell membrane.

The term “intracellular effector domain” (also referred to as the “signaling domain”) as used herein refers to the domain in the CAR that is responsible for intracellular signaling following the binding of the antigen binding domain to the target. The intracellular effector domain is responsible for the activation of at least one of the normal effector functions of the immune cell in which the CAR is expressed. For example, the effector function of a T cell can be a cytolytic activity or helper activity including the secretion of cytokines.

It will be appreciated by a person skilled in the art that VH and/or VL domains disclosed herein may be incorporated, e.g., in the form of a scFv, into CAR-T therapeutics.

### Immunoconjugates

In some embodiments, the anti-BCMA antigen binding protein is used in an immunoconjugate. An “immunoconjugate” (interchangeably referred to as an “antibody-drug conjugate,” “ADC,” or “antigen binding protein-drug conjugate”) comprises an anti-BCMA antigen binding protein conjugated to one or more drugs, such as a cytotoxic agent, such as a chemotherapeutic agent, an immunotherapeutic agent, a growth inhibitory agent, a toxin (e.g., a protein toxin, such as an enzymatically active toxin of bacterial, fungal, plant, or animal origin, or fragments thereof), an antiviral agent, a radioactive isotope (i.e., a radioconjugate), an antibiotic, or a small interfering RNA (siRNA).

In some embodiments, an immunoconjugate has the following general structure:

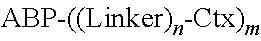

wherein:
ABP is an anti-BCMA antigen binding protein;
Linker is either absent or is any a cleavable or non-cleavable linker;
Ctx is any cytotoxic agent described herein;
n is 0, 1, 2, or 3; and
m is 1, 2, 3, 4, 5, 6, 7, 8, 9 or 10.

Examples of linkers include Val-Cit linkers (e.g., Val-Cit-PAB-OH, Fmoc-Val-Cit-PAB-OH, Fmoc-Val-Cit-PAB-PNP, Boc-Val-Cit, Boc-Val-Cit-PAB, Boc-Val-Cit-PAB-PNP, MC (C5)-Val-Cit, MC-Val-Cit-PAB-OH, MC-Val-Cit-PAB-PNP, SPDP-Val-Cit-PAB-OH, SPDP-Val-Cit-PAB-PNP, Mal-PEG2-Val-Cit-PAB-OH, Mal-PEG4-Val-Cit-PAB-OH, Mal-PEG1-Val-Cit-PAB-PNP, Mal-PEG4-Val-Cit-PAB-PNP, Mal-amido-PEG2-Val-Cit-PAB-OH, Mal-amido-PEG2-Val-Cit-PAB-PNP, Azido-PEG1-Val-Cit-PAB-OH, Azido-PEG3-Val-Cit-PAB-OH, Azido-PEG4-Val-Cit-PAB-OH, Azido-PEG3-Val-Cit-PAB-PNP, BCN-PEG3-Val-Cit, BCN-PEG3-VC-PFP Ester, DBCO-PEG4-Val-Cit-PAB-PNP, TCO-PEG4-Val-Cit-PAB-PNP); Val-Gly linkers (e.g., Boc-Val-Gly-OH, Mal-amido-PEG8-Val-Gly, Mal-amido-PEG8-val-gly-PAB-OH, Boc-Gly-Gly, Fmoc-Gly-Gly-OH, Boc-Gly-Gly-N-[4-(hydroxymethyl)phenyl], Boc-Gly-Gly-Gly-OH, (S)-Benzyl 2-amino-N-[(carboxymethoxy)acetyl]-6-((tert-butoxycarbonyl)amino)hexanoate, Boc-Gly-Gly-Phe-Gly-OH, Fmoc-Gly-Gly-Gly-OH, Gly-Gly-Gly-PEG4-methyltetrazine, Gly-Gly-Gly-PEG3-TCO, Gly-Gly-Gly-PEG4-DBCO, TCO-PEG4-Fmoc-Gly-Gly-Gly, Biotin-PEG11-Gly-Gly-Gly-amine, Biotin-PEG12-Gly-Gly-Gly); Ala-Ala-Asn linkers (e.g., Fmoc-Ala-Pro-OH, Fmoc-Ala-Ser(Psi(Me,Me)pro)-OH, Fmoc-PEG4-Ala-Ala-Asn-PAB, Azido-PEG5-Ala-Ala-Asn-PAB, Fmoc-PEG3-Ala-Ala-Asn(Trt)-PAB, Azido-PEG4-Ala-Ala-Asn(Trt)-PAB, Fmoc-PEG3-Ala-Ala-Asn(Trt)-PAB-PNP); Sulfone-PEG-Acid linkers (e.g., Bis-sulfone-PEG4-Acid, Bis-Sulfone-PEG8-acid, Bis-sulfone-PEG12-Acid, Active-Mono-Sulfone-PEG8-acid); Sulfone-PEG-NHS ester linkers (e.g., Bis-sulfone NHS Ester, Bis-sulfone-PEG4-NHS Ester, Bis-sulfone-PEG8-NHS Ester, Bis-sulfone-PEG12-NHS Ester); PEG Linkers (e.g., Alkyne PEG, Amino PEG, Aminooxy PEG, APN PEG, BCN-PEG, Benzyl-PEG, Biotin PEG, Bis-PEG-acid, Bis-PEG-NHS, Boc-PEG, Branched PEG, Bromo PEG, Cleavable Linkers, DBCO PEG, Diketone Linkers, DNP-PEG, DOTA PEG, Fluorescent reagent, Fmoc PEG, Hydroxy PEG, Iodo PEG, Lipid PEG, m-PEG, Maleimide Linkers, MeNH-PEG, Non-PEG linker, PEG Acid, PEG Aldehyde, PEG Azide, PEG Hydrazide, PEG NHS ester, PEG PFP ester, PEG Phosphonate, PEG Silane, PEG Sulfonic acid, PEG Tosylate, PEG-X-PEG, Peptide Linkers, Poly PEG, Propargyl PEG, PROTAC PEG, SPDP PEG, Sugar PEG, TCO-PEG, Tetrazine-PEG, Thiol PEG); disulfide linkers (e.g., Aminoethyl-SS-ethylalcohol, Aminoethyl-SS-propionic acid, Amino-SS-PEG12-acid, Aminoethyl-SS-ethylamine, t-Boc-Cystamine, Azidoethyl-SS-propionic acid, Azido-SS-PEG2-acid, Azidoethyl-SS-propionic NHS ester, 3-[[2-[(4-azidobenzoyl)amino]ethyl]dithio]propanoicacid, Azido-Phenyl-Amido-S—S-Sulfo-NHS, Azidoethyl-SS-ethylalcohol, Azidoethyl-SS-ethylamine, Azidoethyl-SS-ethylazide, Azido-PEG3-SS-PEG3-azide, Azidoethyl-PEG2-t-Butyl ester, Propargyl-PEG1-SS-alcohol, Propargyl-PEG1-SS-PEG1-acid, Propargyl-PEG1-SS-PEG1-PFP ester, Propargyl-PEG1-SS-PEG1-propargyl, Propargyl-PEG1-SS-PEG1-t-butyl ester, 4-Azide-TFP-Amide-SS-propionic acid, 4-Azide-TFP-Amide-SS-Sulfo-NHS, Acid-PEG2-SS-PEG2-acid, Acid-PEG3-SS-PEG3-acid, Acid-PEG4-S—S-PEG4-acid, Acid-PEG6-SS-PEG6-acid, Boc-NH-ethyl-SS-propionic acid, Boc-aminooxy-ethyl-SS-propanol, Fmoc-NH-ethyl-SS-propionic acid, Fmoc-NH-ethyl-SS-propionic NHS ester, Mal-NH-ethyl-SS-propionic acid, (S)-2-amino-4-(2-(pyridin-2-yl)disulfanyl)butanoic acid, N-(2,2,2-Trifluoroacetyl)-3-[(2-aminoethyl)dithio]propanoic acid, Trifluoroacetamidoethyl-SS-propionic NHS ester, Hydroxy-PEG3-SS-PEG3-alcohol, m-PEG6-SS-PEG6-methyl, THP-SS-alcohol, THP—SS-PEG1-t-butyl ester, THP-SS-PEG1-Tos, 2-hydroxyethyl disulfide mono-Tosylate, Bis-Tos-(2-hydroxyethyl disulfide), Biotin-SS-Amine HCl Salt, Azide-SS-biotin, DBCO-S-S-PEG3-biotin, Biotin-PEG4-S-S-acid, Biotin-bisamido-SS—NHS, NHS-SS-biotin, Sulfo-NHS-SS-biotin, Biotin-PEG4-S-S-NHS, Bis-(Norbornene-PEG23)-disulfides); photocleavable linkers (e.g., PC Biotin-PEG3-alkyne, PC-Biotin-PEG4-PEG4-alkyne, PC Biotin-PEG3-azide, PC-Biotin-PEG4-PEG3-azide, PC Biotin-PEG3-NHS carbonate ester, PC-Biotin-PEG4, PC-Biotin-PEG4-NHS carbonate, PC DBCO-PEG3-biotin, PC SPDP-NHS carbonate ester, PC Alkyne-PEG4-NHS carbonate ester, PC Mal-NHS carbonate ester, PC Azido-PEG3-NHS carbonate ester, PC Azido-PEG11-NHS carbonate ester, 4-Azide-TFP-Amide-PEG4-acid); and enzymatically cleavable linkers (e.g., Mal-PEG2-Val-Cit-PAB-OH, Mal-PEG4-Val-Cit-PAB-OH, Mal-PEG1-Val-Cit-PAB-PNP, Mal-PEG4-Val-Cit-PAB-PNP, Mal-amido-PEG2-Val-Cit-PAB-OH, Mal-amido-PEG2-Val-Cit-PAB-PNP, Azido-PEG1-Val-Cit-PAB-OH, Azido-PEG3-Val-Cit-PAB-OH, Azido-PEG4-Val-Cit-PAB-OH, Azido-PEG3-Val-Cit-PAB—PNP, BCN-PEG3-Val-Cit, BCN-PEG3-VC-PFP Ester, DBCO-PEG4-Val-Cit-PAB-PNP, TCO-PEG4-Val-Cit-PAB-PNP).

In some embodiments, the linker is 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio)pentanoate (SPP), N-succinimidyl 4-(N-maleimidomethyl)cyclohexane-1 carboxylate (SMCC), or N-succinimidyl (4-iodo-acetyl) aminobenzoate (SIAB).

In some embodiments, the immunoconjugate comprises an anti-BCMA monoclonal antibody linked to a cytotoxin. Examples of cytotoxins include Monomethyl auristatin E (MMAE), D8-MMAE, MMAF hydrochloride, PF-06380101, D8-MMAF hydrochloride, Dolastatin 10, MMAF sodium, Auristatin E, MMAF-OMe, MMAF, PF-06380101 D8, Calicheamicin, SN-38, Camptothecin, Dxd, 7-MAD-MDCPT, Top1 inhibitor 1, Doxorubicin hydrochloride, Daunorubicin hydrochloride, Aldoxorubicin, PNU-159682, DaunO2, Daunorubicin, PNU-159682 carboxylic acid, DMEA-PNU-159682, Duocarmycin DM, Duocarmycin DM free base, Duocarmycin™, DC1SMe Duocarmycin A, Duocarmycin SA, DC1, (+)-CBI-CDP12, Duocarmycin Analog, Duocarmycin MB, SG3199, Py-MPB-amino-C3-PBD, Aniline-MPB-amino-C3-PBD, Dimethyl-SGD-1882, Paclitaxel, Methotrexate, α-Amanitin, Taltobulin, Taltobulin hydrochloride, Taltobulin trifluoroacetate, 10-Deacetyl7-xylosyl paclitaxel, C11, β-Amanitin, Tubulysin A, and Telomestatin.

In some embodiments, the immunoconjugate comprises an anti-BCMA monoclonal antibody linked to dovaline-valine-dolaisoleunine-dolaproine-phenylalanine (MMAF) or monomethyl auristatin E (MMAE). In other embodiments, the immunoconjugate comprises a monoclonal antibody linked to MMAF or MMAE by a maleimidocaproyl (MC) linker as depicted in the following structures:

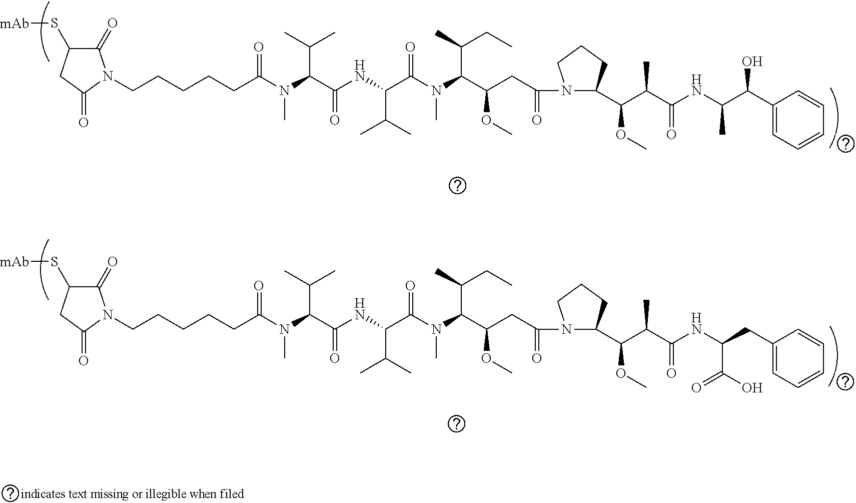

In some embodiments, the immunoconjugate is belantamab mafodotin. Belantamab mafodotin comprises an anti-B CMA-antibody conjugated to a microtubule inhibitor (monomethyl auristatin F, MMAF) via a maleimidocaproyl linker, and is described in the U.S. Prescribing Information for BLENREP® (belantamab mafodotin-blmf). As described in the current prescribing information, the recommended dose for BLENREP® is 2.5 mg/kg as an intravenous infusion over approximately 30 minutes once every 3 weeks. In some embodiments, a combination therapy comprises belantamab mafodotin at a dose of at least about 0.5 mg/kg, 1.0 mg/kg, 1.25 mg/kg, 1.7 mg/kg, 1.92 mg/kg, or 4.6 mg/kg. In some embodiments, a subject is administered an anti-BCMA antigen binding protein at a dose of 1.9 mg/kg Q3/4W, 1.4 mg/kg Q6/8W; 1.9 mg/kg Q6/8W; 1.0 mg/kg Q3/4W, 1.4 mg/kg Q3/4W, 1.4 mg/kg Q9W, 1.4 mg/kg Q12W or 1.0 Q12W.

### Immunomodulatory Imide Drugs

Examples of IMiDs include, but are not limited to, thalidomide (e.g., THALOMID®, lenalidomide (e.g., REVLIMID®), and pomalidomide (e.g., POMALYST®).

### Corticosteroids

Examples of corticosteroids include, but are not limited to, dexamethasone (e.g., DECADRON®, DEXASONE®, DIODEX®, HEXADROL®, MAXIDEX®), prednisone (e.g., DELTASONE®), and methylprednisolone (e.g., MEDROL®).

### Anti-CD38 Antibodies

Examples of anti-CD38 antibodies include, but are not limited to, isatuximab or isatuximab-irfc (e.g., SARCLISA®) and daratumumab (e.g, DARZALEX®, DARZALEX FASPRO®).

### Proteasome Inhibitors

Examples of proteasome inhibitors include, but are not limited to, bortezomib (e.g., VELCADE®), ixazomib (e.g., NINLARO®), carfilzomib (e.g., KYPROLIS®), oprozomib, and delanzomib.

### Gamma-Secretase Inhibitors

Examples of gamma-secretase inhibitors include, but are not limited to, nirogacestat (PF-0308014), crenigacestat (LY3039478), CB-103, tarenflurbil, semagacestat (LY450139), RG-4733, EVP-0962, avagacestat, MK-0752, and BMS-906024, as well as derivatives and polymorphs thereof.

In some embodiments, the gamma-secretase inhibitor is nirogacestat. Nirogacestat, (S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentamide, has the chemical structure of:

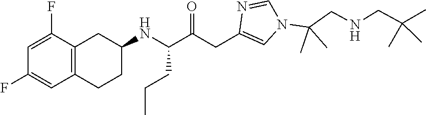 [SMILES: CCC[C@H](N[C@H]1CCc2cc(F)cc(F)c2C1)C(=O)Cc1cn(C(C)(C)CNCC(C)(C)C)cn1]

Form A polymorph of nirogacestat dihydrobromide (a dihydrobromide salt of (S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentanamide of Formula (I))

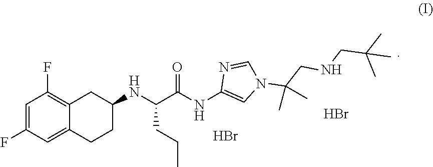 [SMILES: CCC[C@H](N[C@H]1CCc2cc(F)cc(F)c2C1)C(=O)Nc1cn(C(C)(C)CNCC(C)(C)C)cn1 ; Br ; Br]

Form A of nirogacestat dihydrobromide is characterized by an XRPD pattern having peaks at 8.8±0.2, 9.8±0.2, and 23.3±0.2 degrees two theta.

In one aspect, crystalline Form A of nirogacestat dihydrobromide is anhydrous. In another aspect, the melting point of crystalline Form A of nirogacestat dihydrobromide is about 254° C.

In another aspect, Form A of nirogacestat dihydrobromide is characterized by an XRPD pattern having peaks at 8.8±0.2, 9.8±0.2, and 23.3±0.2 degrees two theta when measured by Cu Ka radiation. In another aspect, Form A of nirogacestat dihydrobromide is characterized by an XRPD pattern having peaks at 8.8±0.2, 9.8±0.2, 23.3±0.2, 25.4±0.2, 28.0±0.2, and 29.3±0.2 degrees two theta when measured by Cu Ka radiation.

In another aspect, Form A of nirogacestat dihydrobromide is characterized by an XRPD pattern having peaks at 8.8±0.2, 9.8±0.2, 20.0 0.2, 23.3±0.2, 25.4±0.2, 28.0±0.2, 29.3±0.2, and 32.5±0.2 degrees two theta when measured by Cu Ka radiation.

### Examples of Combination Therapies and their Uses

### Combination Therapies for Newly Diagnosed Multiple Myeloma

In some embodiments, a combination therapy is administered to treat patients newly diagnosed with multiple myeloma. In some of these embodiments, the patients are ineligible for autologous stem cell transplant. In some embodiments, a second combination therapy is administered to the treated patients as maintenance therapy.

“Treating” as used herein refers to administering one or more active agents with the purpose or intent to alleviate, relieve, ameliorate, improve or affect alleviating one or more symptoms or effects associated with a disorder, such as multiple myeloma, and/or slowing the progression of the disorder, such as multiple myeloma. For example, in some embodiments, the disclosed combination therapies reduce ocular toxicity (e.g., changes in corneal epithelium, dry eyes, irritation, redness, blurred vision, photophobia, changes in visual acuity) as compared to administering a therapeutically effective amount of the anti-BCMA antigen binding protein (e.g., belantamab mafodotin). Detection of ocular toxicity may be determined by ophthalmic examination by an ophthalmologist or optometrist, before, during, and/or after treatment.

An ophthalmic examination may include one or more of the following:
1. Best corrected visual acuity,
2. Documentation of manifest refraction and the method used to obtain best corrected visual acuity,
3. Current glasses prescription (if applicable),
4. Intraocular pressure measurement,
5. Anterior segment (slit lamp) examination including fluorescein staining of the cornea and lens examination,
6. Dilated funduscopic examination, and/or
7. An ocular surface disease index (OSDI) which is visual function questionnaire that assess the impact of potential ocular change in vision on function and health-related quality of life.

In some embodiments, reducing ocular toxicity refers to reducing the severity of a corneal adverse reaction or the grade of a treatment related corneal toxicity as determined according to the KVA scale.

| Grade | Corneal Findings | Change in Snellen-Equivalent BCVA |
|---|---|---|
| 0 | Clear cornea | First visit or no change from baseline |
| 1 | Mild (nonconfluent) SPK | VA has decreased not more than |
| | | 0.1 logMAR units from baseline |
| 2 | Presence of | VA has decreased more than |
| | peripheral confluent | 0.1 logMAR units, but not |
| | SPK, patchy | more than 0.3 logMAR units, |
| | microcyst-like | from baseline and is equal |
| | deposits, peripheral | to or better than 1.0 logMAR |
| | subepithelial | (20/200) |
| | haze, or peripheral | |
| | stromal opacity | |
| 3 | Presence of central | VA has decreased more than 0.3 |
| | confluent SPK, | logMAR units from baseline and |
| | diffuse (confluent) | is equal to or better than 1.0 |
| | microcyst-like | logMAR (20/200) |
| | deposits, central | |
| | subepithelial haze, | |
| | or central stromal | |
| | opacity | |
| 4 | Corneal ulcer | VA is worse than 1.0 logMAR |
| | | (20/200) |

Abbreviations: BCVA = best corrected visual acuity; KVA = keratopathy visual acuity; logMAR = logarithm of the minimum angle of resolution; SPK = superficial punctate keratitis.

In some embodiments, as described in more detail below, a patient is treated with a combination therapy comprising a BCMA antigen binding protein, e.g., belantamab mafodotin; a proteasome inhibitor, e.g., bortezomib; an IMiD, e.g., lenalidomide; and a corticosteroid, e.g., dexamethasone on a three-week cycle until cycle eight (“induction treatment”). In some embodiments, the length of a cycle is 21 days for induction treatment cycles. In some embodiments, such treatment is followed by a combination therapy comprising the BCMA antigen binding protein, the IMiD, and the corticosteroid on a four-week cycle thereafter (“maintenance treatment”). In some embodiments, the length of a cycle is 28 days for maintenance treatment cycles. In some embodiments, belantamab mafodotin is administered in combination with bortezomib, lenalidomide, and dexamethasone (“VRd”) every three weeks (Q3W), every six weeks (Q6W), or every nine weeks (Q9W) to Cycle 8, and then in combination with lenalidomide and dexamethasone (“Rd”) every four weeks (Q4W), every eight weeks (Q8W), or every 12 weeks (Q12W) thereafter.

In some embodiments, the combination therapy further comprises a gamma-secretase inhibitor, e.g., nirogacestat.

### Administration of Belantamab Mafodotin

In some embodiments, a patient is treated with a 1.9 mg/kg dose of belantamab mafodotin (Q3/4W). In some embodiments, a patient is treated with a 1.9 mg/kg dose of belantamab mafodotin on Day 1 of each cycle.

In some embodiments, a patient is treated with a 1.4 mg/kg dose of belantamab mafodotin (Q6/8W). In some embodiments, a patient is treated with a 1.4 mg/kg dose of belantamab mafodotin on Day 1 of every other cycle.

In some embodiments, a patient is treated with a 1.9 mg/kg dose of belantamab mafodotin (Q6/8W). In some embodiments, a patient is treated with a 1.9 mg/kg dose of belantamab mafodotin on Day 1 of every other cycle.

In some embodiments, a patient is treated with a 1.0 mg/kg dose of belantamab mafodotin (Q3/4W). In some embodiments, a patient is treated with a 1.0 mg/kg dose of belantamab mafodotin on Day 1 of each cycle.

In some embodiments, a patient is treated with a 1.4 mg/kg dose of belantamab mafodotin (Q3/4W). In some embodiments, a patient is treated with a 1.4 mg/kg dose of belantamab mafodotin on Day 1 of each cycle.

In some embodiments, a patient is treated with a 1.4 mg/kg dose of belantamab mafodotin on Day 1 of cycle 1, then a 1.0 mg/kg dose of belantamab mafodotin on Day 1 of every third cycle from cycle 4.

In some embodiments, a patient is treated with a 1.9 mg/kg dose of belantamab mafodotin on Day 1 of cycle 1, then a 1.4 mg/kg dose of belantamab mafodotin on Day 1 of every third cycle from cycle 4.

In some embodiments, a patient is treated with a 1.9 mg/kg dose of belantamab mafodotin on Day 1 of cycle 1 and cycle 4, then a 1.4 mg/kg dose of belantamab mafodotin on Day 1 of every third cycle from cycle 7.

In some embodiments, a patient is treated with a 1.4 mg/kg dose of belantamab mafodotin on Day 1 of cycle 1 and cycle 3, then a 1.0 mg/kg dose of belantamab mafodotin on Day 1 of every third cycle from cycle 6.

In some embodiments, a patient is treated with a 1.0 mg/kg dose of belantamab mafodotin on Day 1 of cycle 1 and cycle 5, then a 1.0 mg/kg dose of belantamab mafodotin on day 1 of every third cycle from cycle 9.

In some embodiments, a patient is treated with a 1.9 milligram/kilogram (mg/kg) three-weekly (Q3W) dose of belantamab mafodotin intravenously (IV) on Day 1 of every 21-day cycle for the first 8 cycles in combination with VRd (bortezomib, lenalidomide, and dexamethasone, administered as described below). From cycle 9 onwards, the patient is treated with a 1.9 mg/kg four-weekly (Q4W) dose of belantamab mafodotin intravenously on Day 1 of every 28-day cycle in combination with Rd (lenalidomide and dexamethasone, administered as described below).

In some embodiments, a patient is treated with a 1.4 mg/kg six-weekly (Q6W) dose of belantamab mafodotin intravenously on Day 1 of every other 21-day cycle for the first 8 cycles in combination with VRd. From cycle 9 onwards, the patient is treated with a 1.4 mg/kg eight-weekly (Q8W) dose of belantamab mafodotin intravenously on Day 1 of every other 28-day cycle in combination with Rd.

In some embodiments, a patient is treated with a 1.9 mg/kg Q6W dose of belantamab mafodotin intravenously on Day 1 of every other 21-day cycle for the first 8 cycles in combination with VRd. From cycle 9 onwards, the patient is treated with a 1.9 mg/kg Q8W dose of belantamab mafodotin intravenously on Day 1 of every other 28-day cycle in combination with Rd.

In some embodiments, a patient is treated with a 1.0 mg/kg Q3W dose of belantamab mafodotin intravenously on Day 1 of every 21-day cycle for the first 8 cycles in combination with VRd. From cycle 9 onwards, the patient is treated with a 1.0 mg/kg Q4W dose of belantamab mafodotin intravenously on Day 1 of every 28-day cycle in combination with Rd.

In some embodiments, a patient is treated with a 1.4 mg/kg Q3W dose of belantamab mafodotin intravenously on Day 1 of every 21-day cycle for the first 8 cycles in combination with VRd. From cycle 9 onwards, the patient is treated with a 1.4 mg/kg Q4W dose of belantamab mafodotin intravenously on Day 1 of every 28-day cycle in combination with Rd.

In some embodiments, a patient is treated with a 1.4 mg/kg dose of belantamab mafodotin intravenously on Day 1 of cycle 1, then at a dose of 1.0 mg/kg belantamab mafodotin on day 1 of every third cycle from cycle 4 for the first 8 cycles, in combination with VRd, wherein each cycle is 21 days. From cycle 9 onwards, the patient is treated with a 1.0 mg/kg dose of belantamab mafodotin intravenously on Day 1 of every third cycle, wherein each cycle is 28 days, in combination with Rd.

In some embodiments, a patient is treated with a 1.9 mg/kg dose of belantamab mafodotin intravenously on Day 1 of cycle 1, then at a dose of 1.4 mg/kg belantamab mafodotin on day 1 of every third cycle from cycle 4 for the first 8 cycles, in combination with VRd, wherein each cycle is 21 days. From cycle 9 onwards, the patient is treated with a 1.4 mg/kg dose of belantamab mafodotin intravenously on Day 1 of every third cycle, wherein each cycle is 28 days, in combination with Rd.

In some embodiments, a patient is treated with a 1.9 mg/kg dose of belantamab mafodotin intravenously on Day 1 of cycle 1 and cycle 4, then at a dose of 1.4 mg/kg belantamab mafodotin on day 1 of every third cycle from cycle 7 for the first 8 cycles, in combination with VRd, wherein each cycle is 21 days. From cycle 9 onwards, the patient is treated with a 1.4 mg/kg dose of belantamab mafodotin intravenously on Day 1 of every third cycle, wherein each cycle is 28 days, in combination with Rd.

In some embodiments, a patient is treated with a 1.4 mg/kg dose of belantamab mafodotin intravenously on Day 1 of cycle 1 and cycle 3, then at a dose of 1.0 mg/kg belantamab mafodotin on day 1 of every third cycle from cycle 6 for the first 8 cycles, in combination with VRd, wherein each cycle is 21 days. From cycle 9 onwards, the patient is treated with a 1.0 mg/kg dose of belantamab mafodotin intravenously on Day 1 of every third cycle, wherein each cycle is 28 days, in combination with Rd.

In some embodiments, a patient is treated with a 1.0 mg/kg dose of belantamab mafodotin intravenously on Day 1 of cycle 1 and cycle 5 for the first 8 cycles, in combination with VRd, wherein each cycle is 21 days. From cycle 9 onwards, the patient is treated with a 1.0 mg/kg dose of belantamab mafodotin intravenously on Day 1 of every third cycle, wherein each cycle is 28 days, in combination with Rd.

In some embodiments, a patient is treated with either 1.9 mg/kg or 2.5 mg/kg Q9W dose of belantamab mafodotin intravenously on Day 1 of every third 21-day cycle for the first 8 cycles in combination with VRd. From cycle 9 onwards, the patient is treated with either 1.9 mg/kg or 2.5 mg/kg Q12W dose of belantamab mafodotin intravenously on Day 1 of every third 28-day cycle in combination with Rd.

In some embodiments, a patient is treated with a total dose of either 1.9 mg/kg or 2.5 mg/kg of belantamab mafodotin intravenously (split in to two equal doses of 0.95 mg/kg or 1.25 mg/kg to be given on Day 1 and Day 8) Q6W of every other 21-day cycle for the first 8 cycles in combination with VRd. From cycle 9 onwards, the patient is treated with either 1.9 mg/kg or 2.5 mg/kg of belantamab mafodotin intravenously (split in to two equal doses of 0.95 mg/kg or 1.25 mg/kg to be given on Day 1 and Day 8) Q8W of every other 28-day cycle in combination with Rd.

In some embodiments, a patient is treated with 2.5 mg/kg Q6W dose of belantamab mafodotin intravenously on Day 1 of every other 21-day cycle for the first 8 cycles in combination with VRd. From cycle 9 onwards, the patient is treated with 2.5 mg/kg Q8W dose of belantamab mafodotin intravenously on Day 1 of every other 28-day cycle in combination with Rd.

Specifications for belantamab mafodotin treatment are shown in Table 1.

TABLE 1
Specifications for belantamab mafodotin treatment.

| | |
|---|---|
| Dosage form | Lyophilized powder for reconstitution |
| Unit dose | 100 mg/0.75 mg/kg |
| strength/Dose level | 100 mg/1.0 mg/kg |
| | 100 mg/1.4 mg/kg |
| | 100 mg/1.9 mg/kg |
| Route/Administration/Duration | Intravenous infusion/30 min to 1 hour |
| Dosing instructions | Reconstitute belantamab mafodotin for |
| | injection, 100 mg with 2.0 mL of |
| | sterile water for injection, diluted |
| | with saline before use. |
| | Dilute belantamab mafodotin in normal |
| | 0.9% saline to the appropriate |
| | concentration for the dose. Doses of |
| | belantamab mafodotin are administrated |
| | as an IV infusion via an infusion pump. |

In some embodiments, when administered on Day 1, belantamab mafodotin is administered as the first agent in the clinic (prior to VRd or Rd) as a full single dose on Day 1 for 30-60 minute infusion, followed by a 1 to 2 hour rest period. The bortezomib dose is administered approximately 1 hour after belantamab mafodotin infusion is complete.

For patients treated on Day 1 of every cycle (Q3/4W), if a planned dose of belantamab mafodotin is held/missed for any reason, the next dose can be administered at Day 1 of the next planned 21-day or 28-day cycle, as long as the interval between 2 consecutive doses is at least 21 (±3) days or 28 (±3) days, respectively.

For patients treated on Day 1 of every other cycle (Q6/8W), if a planned dose of belantamab mafodotin is held/missed for any reason, the next dose can be administered at Day 1 of the next planned 21-day or 28-day cycle, as long as the interval between 2 consecutive doses is at least 42 (±3) days or 56 (±3) days, respectively.

For patients treated on Day 1 of every third cycle (Q9/12W), if a planned dose of belantamab mafodotin is held/missed for any reason, the next dose can be administered at Day 1 of the next planned 21-day or 28-day cycle, as long as the interval between 2 consecutive doses is at least 63 (±3) days or 84 (±3) days, respectively.

The belantamab mafodotin dose for both induction and maintenance treatment in combination with VRd is based on actual body weight calculated at baseline (Cycle 1 Day 1). If the change in body weight is greater than 10%, the dose can be recalculated based on the actual body weight at the time of dosing.

### Administration of Bortezomib

Bortezomib for injection is supplied in 10 mL vials containing 3.5 mg of bortezomib as a white to off-white cake or powder. The starting dose for bortezomib is 1.3 mg/m². Dose reductions and interruptions are permitted per bortezomib labels [e.g. VELCADE® SmPC, 2021; VELCADE® USPI, 2019]. Decisions regarding dose reduction or modification can be made by the treating physician.

On days when belantamab mafodotin is administered, subcutaneous bortezomib is administered approximately 1 hour after the belantamab mafodotin infusion is complete, assuming the patient is clinically stable. In patients who experience an infusion-related reaction during or after belantamab mafodotin administration, the administration of bortezomib is delayed until the IRR has resolved and the patient is considered clinically stable.

At least 72 hours elapses between consecutive doses of bortezomib. If a bortezomib dose is delayed, subsequent doses are adjusted to account for delay, as all bortezomib doses must be at least 72 hrs apart. Doses that need to be withheld are skipped and are not made up later in the cycle. Individual doses within a cycle have a ±1-day window if beyond 72 hours after last dose. Sites for each SC injection are rotated. New injections are given at least 2.5 cm (1 inch) from an old site and into areas where the site is tender, bruised, erythematous, or indurated. If local injection site reactions occur following administration of bortezomib SC, a less concentrated solution (1 mg/mL instead of 2.5 mg/mL) may be administered SC. Alternatively, substitution to the IV route of administration could be considered for patients who are intolerant to subcutaneous injections or have developed significant edema at potential SC injection sites.

Bortezomib dosing is based on the patient's body surface area (BSA). The reconstituted concentration of bortezomib for subcutaneous administration (2.5 mg/mL) is greater than the reconstituted concentration of bortezomib for intravenous administration (1 mg/mL). After determining the patient's BSA in m², the following equations can be used to calculate the total volume (mL) of reconstituted bortezomib to be administered. For subcutaneous administration (2.5 mg/mL concentration): [bortezomib dose (mg/m²)×patient BSA (m²)]÷[2.5 mg/mL]=Total bortezomib volume (mL) to be administered. For intravenous administration (1 mg/mL concentration): [bortezomib dose (mg/m²)×patient BSA (m²)]÷[1 mg/mL]=Total bortezomib volume (mL) to be administered.

### Administration of Lenalidomide

Lenalidomide is available in 5 mg, 10 mg, 15 mg, 20 mg, and 25 mg capsules for oral administration. Details of pharmaceutical presentation can be found in the representative information for lenalidomide. Lenalidomide is administered at a fixed dose level of 25 or 10 mg orally, depending upon renal function. Patients with an estimated glomerular filtration rate (eGFR) >60 mL/min/1.73 m² receive 25 mg. Patients with eGFR 30-60 mL/min/1.73 m² receive 10 mg.

### Administration of Dexamethasone

Dexamethasone is available as tablets for oral administration. Details of the pharmaceutical presentation can be found in the representative information for dexamethasone. Dexamethasone is administered orally at a fixed dose level of 20 mg in Cycles 1-8 and 40 mg in Cycle 9 onward. Dexamethasone is taken at the same time of the day and may be taken at home. For patients who are older than 75 years, underweight (BMI <18.5), have poorly controlled diabetes mellitus, or prior intolerance/AE to steroid therapy, the dexamethasone dose may be administered at a dose of 10 mg (Cycles 1-8) and 20 mg (Cycle 9+) on the days indicated above.

### Administration of Nirogacestat

Independently, nirogacestat can be administered as part of any of the combination therapies described above. A dose of nirogacestat can be, for example, at least about 50, 100, 150, 200, or 250 mg, administered once or twice daily. In some embodiments, 100 mg of nirogacestat is administered twice a day.

Combination therapies of anti-BCMA antigen binding proteins and gamma-secretase inhibitors, including dosages, duration of treatment, and time lapses between administration of the two agents, are disclosed in WO 2020/208572. In some embodiments, a combination therapy nirogacestat at a dose of at least about 50, 100, 150, 200, or 250 mg.

### Methods of Treating Newly Diagnosed Multiple Myeloma

The disclosure provides methods of treating multiple myeloma in a subject. In some embodiments, the subject is newly diagnosed with multiple myeloma. In some of these embodiments, the subject is ineligible for autologous stem cell transplant.

In some embodiments, the methods comprise administering to the subject: (a) a therapeutically effective dose of an anti-BCMA antigen binding protein; (b) a therapeutically effective dose of a proteasome inhibitor; (c) a therapeutically effective dose of an immunomodulatory imide drug; and (d) a therapeutically effective amount of a corticosteroid.

In some embodiments, the anti-BCMA antigen binding protein is belantamab mafodotin, and wherein the belantamab mafodotin is administered intravenously to the subject according to a schedule selected from the group consisting of: (i) a 1.9 mg/kg dose of belantamab mafodotin on day 1 of every cycle for eight induction treatment cycles, wherein each induction treatment cycle is 21 days; (ii) a 1.4 mg/kg dose of belantamab mafodotin on day 1 of every other cycle for eight induction treatment cycles, wherein each induction treatment cycle is 21 days; (iii) a 1.9 mg/kg dose of belantamab mafodotin on day 1 of every other cycle for eight induction treatment cycles, wherein each induction treatment cycle is 21 days; (iv) a 1.0 mg/kg dose of belantamab mafodotin on day 1 of every cycle for eight induction treatment cycles, wherein each induction treatment cycle is 21 days; (v) a 1.4 mg/kg dose of belantamab mafodotin on day 1 of every cycle for eight induction treatment cycles, wherein each induction treatment cycle is 21 days; (vi) a 1.4 mg/kg dose of belantamab mafodotin on day 1 of cycle 1 and a 1.0 mg/kg dose of belantamab mafodotin on day 1 of every third cycle from cycle 4 for eight induction treatment cycles, wherein each induction treatment cycle is 21 days; (vii) a 1.9 mg/kg dose of belantamab mafodotin on day 1 of cycle 1 and a 1.4 mg/kg dose of belantamab mafodotin on day 1 of every third cycle from cycle 4 for eight induction treatment cycles, wherein each induction treatment cycle is 21 days; (viii) a 1.9 mg/kg dose of belantamab mafodotin on day 1 of cycle 1 and cycle 4 and a 1.4 mg/kg dose of belantamab mafodotin on day 1 of every third cycle from cycle 7 for eight induction treatment cycles, wherein each induction treatment cycle is 21 days; (ix) a 1.4 mg/kg dose of belantamab mafodotin on day 1 of cycle 1 and cycle 3 and a 1.0 mg/kg dose of belantamab mafodotin on day 1 of every third cycle from cycle 6 for eight induction treatment cycles, wherein each induction treatment cycle is 21 days; and (x) a 1.0 mg/kg dose of belantamab mafodotin on day 1 of cycle 1 and cycle 5 for eight induction treatment cycles, wherein each induction treatment cycle is 21 days; wherein the proteasome inhibitor is bortezomib, and wherein the bortezomib is administered to the subject at a dose of 1.3 mg/m² subcutaneously (SC) on days 1, 4, 8, and 11 of every 21-day cycle for eight induction treatment cycles; wherein the immunomodulatory imide drug is lenalidomide, and wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and wherein the corticosteroid is dexamethasone, and wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

In some embodiments, the methods further comprise administering to the subject, after the eight induction treatment cycles, a maintenance therapy comprising: (a) a therapeutically effective dose of an anti-BCMA antigen binding protein; (b) a therapeutically effective dose of an immunomodulatory imide drug; and (c) a therapeutically effective amount of a corticosteroid. In some embodiments, the maintenance therapy does not comprise a proteasome inhibitor. In some embodiments, the maintenance therapy does not comprise bortezomib.

In some embodiments of the maintenance therapy, the anti-BCMA antigen binding protein is belantamab mafodotin, and the belantamab mafodotin is administered intravenously according to a schedule selected from the group consisting of: (i) administration on day 1 of every 28-day cycle; (ii) administration on day 1 of every other 28-day cycle; and (iii) administration on day 1 of every third 28-day cycle; wherein the immunomodulatory imide drug is lenalidomide, and the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and wherein the corticosteroid is dexamethasone, and the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.9 mg/kg on day 1 of every cycle for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.9 mg/kg on day 1 of every cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.4 mg/kg on day 1 of every other cycle for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.4 mg/kg on day 1 of every other cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.9 mg/kg on day 1 of every other cycle for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.9 mg/kg on day 1 of every other cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.0 mg/kg on day 1 of every cycle for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.0 mg/kg on day 1 of every cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.4 mg/kg on day 1 of every cycle for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.4 mg/kg on day 1 of every cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.4 mg/kg on day 1 of cycle 1 and at a dose of 1.0 mg/kg on day 1 of cycle 4 and cycle 7, wherein each treatment cycle is 21 days as an induction treatment, then administering belantamab mafodotin intravenously to the subject at a dose of 1.0 mg/kg on day 1 of every third cycle from cycle 9, wherein each treatment cycle is 28 days as a maintenance treatment. In some embodiments, the methods comprise administering to a subject belantamab mafodotin, wherein the belantamab mafodotin is administered intravenously to the subject at a dose of 1.4 mg/kg on day 1 of cycle 1 and at a dose of 1.0 mg/kg on day 1 of every third cycle from cycle 4 for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.0 mg/kg on day 1 of every third cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.9 mg/kg on day 1 of cycle 1 and at a dose of 1.4 mg/kg on day 1 of cycle 4 and cycle 7, wherein each treatment cycle is 21 days as an induction treatment, then administering belantamab mafodotin intravenously to the subject at a dose of 1.4 mg/kg on day 1 of every third cycle from cycle 9, wherein each treatment cycle is 28 days as a maintenance treatment. In some embodiments, the methods comprise administering to a subject belantamab mafodotin, wherein the belantamab mafodotin is administered intravenously to the subject at a dose of 1.9 mg/kg on day 1 of cycle 1 and at a dose of 1.4 mg/kg dose on day 1 of every third cycle from cycle 4 for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.4 mg/kg on day 1 of every third cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.9 mg/kg on day 1 of cycle 1 and cycle 4, then at a dose of 1.4 mg/kg on day 1 of cycle 7, wherein each treatment cycle is 21 days as an induction treatment, then administering belantamab mafodotin intravenously to the subject at a dose of 1.4 mg/kg on day 1 of every third cycle from cycle 9, wherein each treatment cycle is 28 days as a maintenance treatment. In some embodiments, the methods comprise administering to a subject belantamab mafodotin, wherein the belantamab mafodotin is administered intravenously to the subject at a dose of 1.9 mg/kg on day 1 of cycle 1 and cycle 4 and at a dose of 1.4 mg/kg dose on day 1 of every third cycle from cycle 7 for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.4 mg/kg on day 1 of every third cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.4 mg/kg on day 1 of cycle 1 and cycle 3, then at a dose of 1.0 mg/kg on day 1 of cycle 6, wherein each treatment cycle is 21 days as an induction treatment, then administering belantamab mafodotin intravenously to the subject at a dose of 1.0 mg/kg on day 1 of every third cycle from cycle 9, wherein each treatment cycle is 28 days as a maintenance treatment. In some embodiments, the methods comprise administering to a subject belantamab mafodotin, wherein the belantamab mafodotin is administered intravenously to the subject at a dose of 1.4 mg/kg on day 1 of cycle 1 and cycle 3 and at a dose of 1.0 mg/kg dose on day 1 of every third cycle from cycle 6 for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.0 mg/kg on day 1 of every third cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the methods comprise administering to a subject belantamab mafodotin, wherein the belantamab mafodotin is administered intravenously to the subject at a dose of 1.0 mg/kg on day 1 of cycle 1 and cycle 5, wherein each treatment cycle is 21 days as an induction treatment, then administering belantamab mafodotin intravenously to the subject at a dose of 1.0 mg/kg on day 1 of every third cycle from cycle 9, wherein each treatment cycle is 28 days as a maintenance treatment. In some embodiments, the methods comprise administering to a subject belantamab mafodotin, wherein the belantamab mafodotin is administered intravenously to the subject at a dose of 1.0 mg/kg on day 1 of cycle 1 and cycle 5 for eight induction treatment cycles, wherein each induction treatment cycle is 21 days; bortezomib, wherein the bortezomib is administered to the subject at a dose of 1.3 mg/m² subcutaneously (SC) on days 1, 4, 8, and 11 of every 21-day cycle for eight induction treatment cycles; lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further administering to the subject, after the eight induction treatment cycles, a maintenance therapy comprising: belantamab mafodotin, wherein the belantamab mafodotin is administered intravenously to the subject at a dose of 1.0 mg/kg on day 1 of every third cycle from cycle 9 onwards, wherein each cycle is 28 days; lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

The disclosure also provides the use of: (a) a therapeutically effective dose of an anti-BCMA antigen binding protein; (b) a therapeutically effective dose of a proteasome inhibitor; (c) a therapeutically effective dose of an immunomodulatory imide drug; and (d) a therapeutically effective amount of a corticosteroid in the manufacture of a medicament for use in treating multiple myeloma in a subject. The disclosure also provides: (a) a therapeutically effective dose of an anti-BCMA antigen binding protein; (b) a therapeutically effective dose of a proteasome inhibitor; (c) a therapeutically effective dose of an immunomodulatory imide drug; and (d) a therapeutically effective amount of a corticosteroid for use in treating multiple myeloma in a subject. In some embodiments the subject is newly diagnosed with multiple myeloma. In some of these embodiments, the subject is ineligible for autologous stem cell transplant.

In some embodiments, the anti-BCMA antigen binding protein is belantamab mafodotin, and wherein the belantamab mafodotin is formulated for intravenous administration to the subject according to a schedule selected from the group consisting of: (i) a 1.9 mg/kg dose of belantamab mafodotin on day 1 of every cycle for eight induction treatment cycles, wherein each induction treatment cycle is 21 days; (ii) a 1.4 mg/kg dose of belantamab mafodotin on day 1 of every other cycle for eight induction treatment cycles, wherein each induction treatment cycle is 21 days; (iii) a 1.9 mg/kg dose of belantamab mafodotin on day 1 of every other cycle for eight induction treatment cycles, wherein each induction treatment cycle is 21 days; (iv) a 1.0 mg/kg dose of belantamab mafodotin on day 1 of every cycle for eight induction treatment cycles, wherein each induction treatment cycle is 21 days; (v) a 1.4 mg/kg dose of belantamab mafodotin on day 1 of every cycle for eight induction treatment cycles, wherein each induction treatment cycle is 21 days; (vi) a 1.4 mg/kg dose of belantamab mafodotin on day 1 of cycle 1 and a 1.0 mg/kg dose of belantamab mafodotin on day 1 of every third cycle from cycle 4 for eight induction treatment cycles, wherein each induction treatment cycle is 21 days; (vii) a 1.9 mg/kg dose of belantamab mafodotin on day 1 of cycle 1 and a 1.4 mg/kg dose of belantamab mafodotin on day 1 of every third cycle from cycle 4 for eight induction treatment cycles, wherein each induction treatment cycle is 21 days; (viii) a 1.9 mg/kg dose of belantamab mafodotin on day 1 of cycle 1 and cycle 4 and a 1.4 mg/kg dose of belantamab mafodotin on day 1 of every third cycle from cycle 7 for eight induction treatment cycles, wherein each induction treatment cycle is 21 days; (ix) a 1.4 mg/kg dose of belantamab mafodotin on day 1 of cycle 1 and cycle 3 and a 1.0 mg/kg dose of belantamab mafodotin on day 1 of every third cycle from cycle 6 for eight induction treatment cycles, wherein each induction treatment cycle is 21 days; and (x) a 1.0 mg/kg dose of belantamab mafodotin on day 1 of cycle 1 and cycle 5 for eight induction treatment cycles, wherein each induction treatment cycle is 21 days; wherein the proteasome inhibitor is bortezomib, and wherein the bortezomib is formulated for subcutaneous (SC) administration to the subject at a dose of 1.3 mg/m² on days 1, 4, 8, and 11 of every 21-day cycle for eight induction treatment cycles; wherein the immunomodulatory imide drug is lenalidomide, and wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and wherein the corticosteroid is dexamethasone, and wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

In some embodiments, the use further comprises a maintenance therapy for administration to the subject after the eight induction treatment cycles, comprising: (a) a therapeutically effective dose of an anti-BCMA antigen binding protein; (b) a therapeutically effective dose of an immunomodulatory imide drug; and (c) a therapeutically effective amount of a corticosteroid. In some embodiments, the maintenance therapy does not comprise a proteasome inhibitor. In some embodiments, the maintenance therapy does not comprise bortezomib.

In some embodiments of the maintenance therapy, the anti-BCMA antigen binding protein is belantamab mafodotin, and the belantamab mafodotin is formulated for intravenous administration according to a schedule selected from the group consisting of: (i) administration on day 1 of every 28-day cycle; (ii) administration on day 1 of every other 28-day cycle; and (iii) administration on day 1 of every third 28-day cycle; wherein the immunomodulatory imide drug is lenalidomide, and the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and wherein the corticosteroid is dexamethasone, and the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.9 mg/kg on day 1 of every cycle for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.9 mg/kg on day 1 of every cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.4 mg/kg on day 1 of every other cycle for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.4 mg/kg on day 1 of every other cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.9 mg/kg on day 1 of every other cycle for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.9 mg/kg on day 1 of every other cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.0 mg/kg on day 1 of every cycle for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.0 mg/kg on day 1 of every cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.4 mg/kg on day 1 of every cycle for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further the maintenance therapy comprises: belantamab mafodotin, wherein the belantamab mafodotin is formulated for intravenous administration to the subject at a dose of 1.4 mg/kg on day 1 of every cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.4 mg/kg on day 1 of cycle 1 and at a dose of 1.0 mg/kg on day 1 of every third cycle from cycle 4 for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.0 mg/kg on day 1 of every third cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.9 mg/kg on day 1 of cycle 1 and at a dose of 1.4 mg/kg dose on day 1 of every third cycle from cycle 4 for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.4 mg/kg on day 1 of every third cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.9 mg/kg on day 1 of cycle 1 and cycle 4 and at a dose of 1.4 mg/kg dose on day 1 of every third cycle from cycle 7 for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.4 mg/kg on day 1 of every third cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.4 mg/kg on day 1 of cycle 1 and cycle 3 and at a dose of 1.0 mg/kg dose on day 1 of every third cycle from cycle 6 for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and further the maintenance therapy comprises: belantamab mafodotin, wherein the belantamab mafodotin is formulated for intravenous administration to the subject at a dose of 1.0 mg/kg on day 1 of every third cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

In some embodiments, the induction treatment comprises belantamab mafodotin, wherein the belantamab mafodotin is formulated for intravenous administration to the subject at a dose of 1.0 mg/kg on day 1 of cycle 1 and cycle 5 for eight induction treatment cycles, wherein each induction treatment cycle is 21 days; bortezomib, wherein the bortezomib is formulated for subcutaneous (SC) administration to the subject at a dose of 1.3 mg/m² on days 1, 4, 8, and 11 of every 21-day cycle for eight induction treatment cycles; lenalidomide, wherein the lenalidomide is formulated for oral administration to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and dexamethasone, wherein the dexamethasone is formulated for oral administration to the subject at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and the maintenance therapy comprises: belantamab mafodotin, wherein the belantamab mafodotin is formulated for intravenous administration to the subject at a dose of 1.0 mg/kg on day 1 of every third cycle from cycle 9 onwards, wherein each cycle is 28 days; lenalidomide, wherein the lenalidomide is formulated for oral administration to the subject at a dose of (i) 25 mg on each of days 1-21 of each 28-day cycle or (ii) 10 mg on days 1-21 of each 28-day cycle if the subject has eGFR 30-60 mL/min/1.73 m²; and dexamethasone, wherein the dexamethasone is formulated for oral administration to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

### Example of Dosing Schedules

In some embodiments, the dosing schedule comprises a total of 8 Cycles of belantamab mafodotin+VRd induction treatment followed by belantamab mafodotin+doublet Rd maintenance treatment. In some embodiments, the dosing schedule comprises a total of 8 induction treatment cycles of belantamab mafodotin administered in combination with VRd, wherein each cycle is 21 days, followed by administration of belantamab mafodotin in combination with Rd maintenance treatment, wherein each cycle is 28 days.

### Induction Treatment (Q3W)−Belantamab Mafodotin+VRd; up to Cycle 8

Belantamab mafodotin is administered intravenously (IV) on Day 1, either every 21-day cycle, every other 21-day cycle, every third 21-day cycle, or every fourth 21-day cycle for the first 8 (induction) Cycles in combination with VRd.

In some embodiments, the induction treatment comprises administering belantamab mafodotin at a dose of 1.9 mg/kg on day 1 of every cycle for eight induction treatment cycles, wherein each cycle is 21 days. In some embodiments, the induction treatment comprises administering belantamab mafodotin at a dose of 1.4 mg/kg on day 1 of every other cycle for eight induction treatment cycles, wherein each cycle is 21 days. In some embodiments, the induction treatment comprises administering belantamab mafodotin at a dose of 1.9 mg/kg on day 1 of every other cycle for eight induction treatment cycles, wherein each cycle is 21 days. In some embodiments, the induction treatment comprises administering belantamab mafodotin at a dose of 1.0 mg/kg on day 1 of every cycle for eight induction treatment cycles, wherein each cycle is 21 days. In some embodiments, the induction treatment comprises administering belantamab mafodotin at a dose of 1.4 mg/kg on day 1 of every cycle for eight induction treatment cycles, wherein each cycle is 21 days.

In some embodiments, the induction treatment comprises administering belantamab mafodotin at a dose of 1.4 mg/kg on day 1 of cycle 1, then at a dose of 1.0 mg/kg on day 1 of cycle 4 and cycle 7, wherein each cycle is 21 days. In some embodiments, the induction treatment comprises administering belantamab mafodotin at a dose of 1.9 mg/kg on day 1 of cycle 1, then at a dose of 1.4 mg/kg on day 1 of cycle 4 and cycle 7, wherein each cycle is 21 days.

In some embodiments, the induction treatment comprises administering belantamab mafodotin at a dose of 1.9 mg/kg on day 1 of cycle 1 and cycle 4, then at a dose of 1.4 mg/kg on day 1 of cycle 7, wherein each cycle is 21 days. In some embodiments, the induction treatment comprises administering belantamab mafodotin at a dose of 1.4 mg/kg on day 1 of cycle 1 and cycle 3, then at a dose of 1.0 mg/kg on day 1 of cycle 6, wherein each cycle is 21 days. In some embodiments, the induction treatment comprises administering belantamab mafodotin at a dose of 1.0 mg/kg on day 1 of cycle 1 and cycle 5, wherein each cycle is 21 days.

Bortezomib is administered at a dose of 1.3 mg/m² subcutaneously (SC) on Days 1, 4, 8, and 11 of every 21-day cycle for the eight induction Cycles.

Lenalidomide is administered at a dose of 25 mg, orally, on Days 1-14 of a 21-day cycle for the eight induction Cycles. In patients with eGFR 30-60 mL/min/1.73 m² lenalidomide is administered at 10 mg daily on Days 1-14 of each 21-day cycle. In patients whose renal function improves during treatment to eGFR >60 mL/min/1.73 m² (confirmed, e.g., over two measurements two weeks apart), the dose of lenalidomide can be increased accordingly based on the Investigator judgement.

Dexamethasone is administered at 20 mg, orally, on Days 1, 2, 4, 5, 8, 9, 11, and 12 of a 21-day cycle for the eight induction Cycles. Dexamethasone is taken at the same time of the day and may be taken at home. For patients who are older than 75 years, underweight (body mass index (BMI)<18.5), have poorly controlled diabetes mellitus, or prior intolerance/AE to steroid therapy, the dexamethasone dose may be administered at a dose of 10 mg on the Days indicated above.

### Maintenance Treatment (Q4W): Belantamab Mafodotin+Rd; Cycle 9 Onward

In some embodiments, after the first 8 induction Cycles, belantamab mafodotin in combination with Rd is administered as maintenance treatment. Maintenance treatment follows induction treatment and begins at cycle 9. In some embodiments, each cycle of maintenance treatment is 28 days. Bortezomib is not administered as part of maintenance treatment.

Belantamab mafodotin is administered intravenously (IV) on Day 1, either every 28-day cycle, every other 28-day cycle or every third 28-day cycle in combination with Rd.

In some embodiments, the maintenance treatment comprises administering belantamab mafodotin at a dose of 1.9 mg/kg on day 1 of every cycle from cycle 9 onwards (e.g., cycle 9, cycle 10, cycle 11, cycle 12, and so on), wherein each cycle is 28 days. In some embodiments, the maintenance treatment comprises administering belantamab mafodotin at a dose of 1.4 mg/kg on day 1 of every other cycle from cycle 9 onwards (e.g., cycle 9, cycle 11, cycle 13, cycle 15, and so on), wherein each cycle is 28 days. In some embodiments, the maintenance treatment comprises administering belantamab mafodotin at a dose of 1.9 mg/kg on day 1 of every other cycle from cycle 9 onwards (e.g., cycle 9, cycle 11, cycle 13, cycle 15, and so on), wherein each cycle is 28 days. In some embodiments, the maintenance treatment comprises administering belantamab mafodotin at a dose of 1.0 mg/kg on day 1 of every cycle from cycle 9 onwards (e.g., cycle 9, cycle 10, cycle 11, cycle 12, and so on), wherein each cycle is 28 days. In some embodiments, the maintenance treatment comprises administering belantamab mafodotin at a dose of 1.4 mg/kg on day 1 of every cycle from cycle 9 onwards (e.g., cycle 9, cycle 10, cycle 11, cycle 12, and so on), wherein each cycle is 28 days.

In some embodiments, the maintenance treatment comprises administering belantamab mafodotin at a dose of 1.0 mg/kg on day 1 of every third cycle from cycle 9 (e.g., cycle 9, cycle 12, cycle 15, cycle 18, and so on), wherein each cycle is 28 days. In some embodiments, the maintenance treatment comprises administering belantamab mafodotin at a dose of 1.4 mg/kg on day 1 of every third cycle from cycle 9 (e.g., cycle 9, cycle 12, cycle 15, cycle 18, and so on), wherein each cycle is 28 days.

Lenalidomide is administered at a dose of 25 mg orally on Days 1-21 of each 28-day cycle until PD or unacceptable toxicity. In patients with eGFR 30-60 mL/min/1.73 m² lenalidomide is administered at 10 mg daily on Days 1-21 of each 28-day cycle. In patients whose renal function improves during treatment to eGFR >60 mL/min/1.73 m² (confirmed over 2 measurements 2 weeks apart), the dose of lenalidomide can be increased accordingly based on the Investigator judgement.

Dexamethasone is administered at 40 mg, orally, on Days 1, 8, 15 and 22 of a 28-day cycle from Cycle 9 onward, until PD or unacceptable toxicity. Dexamethasone is taken at the same time of the day and may be taken at home. For patients who are older than 75 years, underweight (BMI <18.5), have poorly controlled diabetes mellitus, or prior intolerance/AE to steroid therapy, the dexamethasone dose may be administered at a dose of 20 mg on the days indicated above.

Independently, the dose of nirogacestat administered as part of any of the combination therapies described above can be, for example, at least about 50, 100, 150, 200, or 250 mg, administered once or twice daily. In some embodiments, 100 mg of nirogacestat is administered twice a day.

### Kits

A kit-of-parts comprising a pharmaceutical composition together with instructions for use is further provided. For convenience, the kit-of-parts may comprise reagents in predetermined amounts with instructions for use.

In some embodiments, disclosed herein are kits comprising the combination therapy disclosed herein. A kit may include a plurality of syringes, ampules, foil packets, or blister packs, each containing a single unit dose of a kit component described herein. Containers of a kit may be airtight, waterproof (e.g., impermeable to changes in moisture or evaporation), and/or light-tight. A kit may include a device suitable for administration of the component, e.g., a syringe, inhalant, pipette, forceps, measured spoon, dropper (e.g., eye dropper), swab (e.g., a cotton swab or wooden swab), or any such delivery device. In some embodiments, the device may be a medical implant device, e.g., packaged for surgical insertion. A kit disclosed herein may comprise one or more reagents or instruments which enable the method to be carried out.

In addition to the above components, instructions for use may be provided in a kit. These instructions may be present in the kit in a variety of forms, such as printed information on a suitable medium or substrate (e.g., a piece or pieces of paper on which the information is printed), in the packaging of the kit, in a package insert, etc. In some embodiments, instructions for use can be provided on a computer readable medium (e.g., jump/thumb drive, CD, etc.), on which the information has been recorded or at a website address which may be used via the internet to access the information at a website.

### Devices

Another aspect of the present disclosure provides a pre-filled syringe or autoinjector device, comprising the combination therapy described herein. In some embodiments, a combination is stored in a container, pre-filled syringe, injector or autoinjector device.

Those skilled in the art will appreciate that there are numerous variations and permutations of the above described embodiments that fall within the scope of the appended claims.

### EXAMPLES

### Example 1

A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.0 mg/kg dose of belantamab mafodotin on Day 1 of cycle 1 and cycle 5 and a 1.0 mg/kg dose of belantamab mafodotin on day 1 of every third cycle from cycle 9; (b) administration of bortezomib at a dose of 1.3 mg/m² subcutaneously (SC) on Days 1, 4, 8, and 11 of every 21-day cycle for eight induction Cycles; (c) administration of lenalidomide (1) at a dose of 25 mg, orally, on Days 1-14 of a 21-day cycle for eight induction Cycles or (2) at 10 mg daily on Days 1-14 of each 21-day cycle if the individual has eGFR 30-60 mL/min/1.73 m²; and (d) administration of dexamethasone orally at a dose of 20 mg on Days 1, 2, 4, 5, 8, 9, 11, and 12 of a 21-day cycle for eight induction Cycles.

An additional therapeutic benefit is provided to the patient to whom this combination therapy is administered in comparison to a patient to whom is administered the standard of care treatment alone.

### Example 2

A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.4 mg/kg dose of belantamab mafodotin on day 1 of cycle 1 and a 1.0 mg/kg dose of belantamab mafodotin on day 1 of every third cycle from cycle 4 for eight induction treatment cycles, wherein each induction treatment cycle is 21 days; (b) administration of bortezomib at a dose of 1.3 mg/m² subcutaneously (SC) on days 1, 4, 8, and 11 of every 21-day cycle for eight induction treatment cycles; (c) administration of lenalidomide orally (1) at a dose of 25 mg on days 1-14 of a 21-day cycle for eight induction treatment cycles or (2) at a dose of 10 mg daily on days 1-14 of each 21-day cycle for eight induction treatment cycles if the individual has eGFR 30-60 mL/min/1.73 m²; and (d) administration of dexamethasone orally at a dose of (i) 20 mg on days 1, 2, 4, 5, 8, 9, 11, and 12 of a 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

The combination therapy further comprises (a) a 1.4 mg/kg dose of belantamab mafodotin on day 1 of every third cycle from cycle 9 onwards, wherein each cycle is 28 days; (b) administration of lenalidomide orally (1) at a dose of 25 mg on days 1-21 of a 28-day cycle from cycle 9 onwards or (2) at a dose of 10 mg daily on days 1-21 of each 28-day cycle if the individual has eGFR 30-60 mL/min/1.73 m²; and (c) administration of dexamethasone orally at a dose of (i) 40 mg on days 1, 8, 15 and 22 of each 28-day cycle from cycle 9 onwards or (ii) 20 mg on days 1, 8, 15, and 22 of each 28-day cycle if the subject is older than 75 years or has a body mass index (BMI) of <18.5.

An additional therapeutic benefit is provided to the patient to whom this combination therapy is administered in comparison to a patient to whom is administered the standard of care treatment alone.

### Example 3

A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.9 mg/kg dose of belantamab mafodotin on day 1 of cycle 1 and a 1.4 mg/kg dose of belantamab mafodotin on day 1 of every third cycle from cycle 4 for eight induction treatment cycles, wherein each induction treatment cycle is 21 days; (b) administration of bortezomib at a dose of 1.3 mg/m² subcutaneously (SC) on days 1, 4, 8, and 11 of every 21-day cycle for eight induction treatment cycles; (c) administration of lenalidomide orally (1) at a dose of 25 mg on days 1-14 of a 21-day cycle for eight induction treatment cycles or (2) at a dose of 10 mg daily on days 1-14 of each 21-day cycle for eight induction treatment cycles if the individual has eGFR 30-60 mL/min/1.73 m²; and (d) administration of dexamethasone orally at a dose of (i) 20 mg on days 1, 2, 4, 5, 8, 9, 11, and 12 of a 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

The combination therapy further comprises (a) a 1.4 mg/kg dose of belantamab mafodotin on day 1 of every third cycle from cycle 9 onwards, wherein each cycle is 28 days; (b) administration of lenalidomide orally (1) at a dose of 25 mg on days 1-21 of a 28-day cycle from cycle 9 onwards or (2) at a dose of 10 mg daily on days 1-21 of each 28-day cycle if the individual has eGFR 30-60 mL/min/1.73 m²; and (c) administration of dexamethasone orally at a dose of (i) 40 mg on days 1, 8, 15 and 22 of each 28-day cycle from cycle 9 onwards or (ii) 20 mg on days 1, 8, 15, and 22 of each 28-day cycle if the subject is older than 75 years or has a body mass index (BMI) of <18.5.

An additional therapeutic benefit is provided to the patient to whom this combination therapy is administered in comparison to a patient to whom is administered the standard of care treatment alone.

### Example 4

A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.9 mg/kg dose of belantamab mafodotin on day 1 of cycle 1 and cycle 4, then a 1.4 mg/kg dose of belantamab mafodotin on day 1 of every third cycle from cycle 7 for eight induction treatment cycles, wherein each induction treatment cycle is 21 days; (b) administration of bortezomib at a dose of 1.3 mg/m² subcutaneously (SC) on days 1, 4, 8, and 11 of every 21-day cycle for eight induction treatment cycles; (c) administration of lenalidomide orally (1) at a dose of 25 mg on days 1-14 of a 21-day cycle for eight induction treatment cycles or (2) at a dose of 10 mg daily on days 1-14 of each 21-day cycle for eight induction treatment cycles if the individual has eGFR 30-60 mL/min/1.73 m²; and (d) administration of dexamethasone orally at a dose of (i) 20 mg on days 1, 2, 4, 5, 8, 9, 11, and 12 of a 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

The combination therapy further comprises (a) a 1.4 mg/kg dose of belantamab mafodotin on day 1 of every third cycle from cycle 9 onwards, wherein each cycle is 28 days; (b) administration of lenalidomide orally (1) at a dose of 25 mg on days 1-21 of a 28-day cycle from cycle 9 onwards or (2) at a dose of 10 mg daily on days 1-21 of each 28-day cycle if the individual has eGFR 30-60 mL/min/1.73 m²; and (c) administration of dexamethasone orally at a dose of (i) 40 mg on days 1, 8, 15 and 22 of each 28-day cycle from cycle 9 onwards or (ii) 20 mg on days 1, 8, 15, and 22 of each 28-day cycle if the subject is older than 75 years or has a body mass index (BMI) of <18.5.

An additional therapeutic benefit is provided to the patient to whom this combination therapy is administered in comparison to a patient to whom is administered the standard of care treatment alone.

### Example 5

A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.4 mg/kg dose of belantamab mafodotin on day 1 of cycle 1 and cycle 3, then a 1.0 mg/kg dose of belantamab mafodotin on day 1 of every third cycle from cycle 6 for eight induction treatment cycles, wherein each induction treatment cycle is 21 days; (b) administration of bortezomib at a dose of 1.3 mg/m² subcutaneously (SC) on days 1, 4, 8, and 11 of every 21-day cycle for eight induction treatment cycles; (c) administration of lenalidomide orally (1) at a dose of 25 mg on days 1-14 of a 21-day cycle for eight induction treatment cycles or (2) at a dose of 10 mg daily on days 1-14 of each 21-day cycle for eight induction treatment cycles if the individual has eGFR 30-60 mL/min/1.73 m²; and (d) administration of dexamethasone orally at a dose of (i) 20 mg on days 1, 2, 4, 5, 8, 9, 11, and 12 of a 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

The combination therapy further comprises (a) a 1.0 mg/kg dose of belantamab mafodotin on day 1 of every third cycle from cycle 9 onwards, wherein each cycle is 28 days; (b) administration of lenalidomide orally (1) at a dose of 25 mg on days 1-21 of a 28-day cycle from cycle 9 onwards or (2) at a dose of 10 mg daily on days 1-21 of each 28-day cycle if the individual has eGFR 30-60 mL/min/1.73 m²; and (c) administration of dexamethasone orally at a dose of (i) 40 mg on days 1, 8, 15 and 22 of each 28-day cycle from cycle 9 onwards or (ii) 20 mg on days 1, 8, 15, and 22 of each 28-day cycle if the subject is older than 75 years or has a body mass index (BMI) of <18.5.

An additional therapeutic benefit is provided to the patient to whom this combination therapy is administered in comparison to a patient to whom is administered the standard of care treatment alone.

### Example 6

A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.0 mg/kg dose of belantamab mafodotin on day 1 of cycle 1 and cycle 5, wherein each induction treatment cycle is 21 days; (b) administration of bortezomib at a dose of 1.3 mg/m² subcutaneously (SC) on days 1, 4, 8, and 11 of every 21-day cycle for eight induction treatment cycles; (c) administration of lenalidomide orally (1) at a dose of 25 mg on days 1-14 of a 21-day cycle for eight induction treatment cycles or (2) at a dose of 10 mg daily on days 1-14 of each 21-day cycle for eight induction treatment cycles if the individual has eGFR 30-60 mL/min/1.73 m²; and (d) administration of dexamethasone orally at a dose of (i) 20 mg on days 1, 2, 4, 5, 8, 9, 11, and 12 of a 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

The combination therapy further comprises (a) a 1.0 mg/kg dose of belantamab mafodotin on day 1 of every third cycle from cycle 9 onwards, wherein each cycle is 28 days; (b) administration of lenalidomide orally (1) at a dose of 25 mg on days 1-21 of a 28-day cycle from cycle 9 onwards or (2) at a dose of 10 mg daily on days 1-21 of each 28-day cycle if the individual has eGFR 30-60 mL/min/1.73 m²; and (c) administration of dexamethasone orally at a dose of (i) 40 mg on days 1, 8, 15 and 22 of each 28-day cycle from cycle 9 onwards or (ii) 20 mg on days 1, 8, 15, and 22 of each 28-day cycle if the subject is older than 75 years or has a body mass index (BMI) of <18.5.

An additional therapeutic benefit is provided to the patient to whom this combination therapy is administered in comparison to a patient to whom is administered the standard of care treatment alone.

1. A combination therapy, comprising:
(a) a therapeutically effective dose of an anti-BCMA antigen binding protein;
(b) a therapeutically effective dose of a proteasome inhibitor;
(c) a therapeutically effective dose of an immunomodulatory imide drug; and
(d) a therapeutically effective amount of a corticosteroid,
wherein the anti-BCMA antigen binding protein is belantamab mafodotin, the proteasome inhibitor is bortezomib, the immunomodulatory imide drug is lenalidomide and the corticosteroid is dexamethasone.

2.-6. (canceled)

7. The combination therapy of claim 61, which comprises:
(a) administration of belantamab mafodotin according to a schedule selected from the group consisting of
(i) 1.9 mg/kg dose of belantamab mafodotin Q3/4W, 1.9 mg/kg on Day 1 of every cycle;
(ii) a 1.4 mg/kg dose of belantamab mafodotin Q6/8W, 1.4 mg/kg on Day 1 of every other cycle;
(iii) a 1.9 mg/kg dose of belantamab mafodotin Q6/8W on Day 1 of every other cycle;
(iv) a 1.0 mg/kg dose of belantamab mafodotin Q3/4W, 1.0 mg/kg on Day 1 of every cycle;
(v) a 1.4 mg/kg dose of belantamab mafodotin Q3/4W, 1.4 mg/kg on Day 1 of every cycle;
(vi) a 1.4 mg/kg dose of belantamab mafodotin on Day 1 of cycle 1 and 1.0 mg/kg on Day 1 of every third cycle from cycle 4;
(vii) a 1.9 mg/kg dose of belantamab mafodotin on Day 1 of cycle 1 and 1.4 mg/kg of belantamab mafodotin on Day 1 of every third cycle from cycle 4;
(viii) a 1.9 mg/kg dose of belantamab mafodotin on Day 1 of cycle 1 and cycle 4 and a 1.4 mg/kg of belantamab mafodotin on Day 1 of every third cycle from cycle 7;
(ix) a 1.4 mg/kg dose of belantamab mafodotin on Day 1 of cycle 1 and cycle 3 and a 1.0 mg/kg dose of belantamab mafodotin on Day 1 of every third cycle from cycle 6; and
(x) a 1.0 mg/kg dose of belantamab mafodotin on Day 1 of cycle 1 and cycle 5 and a 1.0 mg/kg dose of belantamab mafodotin on day 1 of every third cycle from cycle 9;
(b) administration of bortezomib at a dose of 1.3 mg/m² subcutaneously (SC) on Days 1, 4, 8, and 11 of every 21-day cycle for eight induction Cycles;
(c) administration of lenalidomide (1) at a dose of 25 mg, orally, on Days 1-14 of a 21-day cycle for eight induction Cycles or (2) at 10 mg daily on Days 1-14 of each 21-day cycle if the individual has eGFR 30-60 mL/min/1.73 m²; and
(d) administration of dexamethasone orally at a dose of 20 mg on Days 1, 2, 4, 5, 8, 9, 11, and 12 of a 21-day cycle for eight induction Cycles.

8. The combination therapy of claim 7, further comprising a second combination therapy, comprising:
(a) a therapeutically effective dose of an anti-BCMA antigen binding protein;
(b) a therapeutically effective dose of an immunomodulatory imide drug; and
(c) a therapeutically effective amount of a corticosteroid,
wherein the anti-BCMA antigen binding protein is belantamab mafodotin, the immunomodulatory imide drug is lenalidomide, and the corticosteroid is dexamethasone.

9.-12. (canceled)

13. The combination therapy of claim 12, wherein administration of the second combination therapy comprises:
(a) intravenous administration of belantamab mafodotin on a schedule selected from the group consisting of
(i) administration on Day 1 of every 28-day cycle;
(ii) administration on Day 1 of every other 28-day cycle; and
(iii) administration on Day 1 of every third 28-day cycle;
(b) oral administration of lenalidomide at a dose of (i) 25 mg on Days 1-21 of each 28-day cycle or (ii) 10 mg on Days 1-21 of each 28-day cycle; and
(c) oral administration of dexamethasone at a dose of 20 mg or 40 mg on Days 1, 8, 15 and 22 of each 28-day cycle.

14. A method of treating newly diagnosed multiple myeloma, comprising administering to an individual in need thereof a combination therapy of claim 1.

15. The method of claim 14, wherein the individual is ineligible for autologous stem cell transplant.

16. The method of claim 14, further comprising a maintenance therapy comprising a second combination therapy, comprising:
(a) a therapeutically effective dose of an anti-BCMA antigen binding protein;
(b) a therapeutically effective dose of an immunomodulatory imide drug; and
(c) a therapeutically effective amount of a corticosteroid,
wherein the anti-BCMA antigen binding protein is belantamab mafodotin, the immunomodulatory imide drug is lenalidomide, and the corticosteroid is dexamethasone.

17.-20. (canceled)

21. A method of treating multiple myeloma in a subject, comprising administering to the subject:
(a) a therapeutically effective dose of an anti-BCMA antigen binding protein;
(b) a therapeutically effective dose of a proteasome inhibitor;
(c) a therapeutically effective dose of an immunomodulatory imide drug; and
(d) a therapeutically effective amount of a corticosteroid,
wherein the anti-BCMA antigen binding protein is belantamab mafodotin, and wherein the belantamab mafodotin is administered intravenously to the subject according to a schedule selected from the group consisting of:
(i) a 1.9 mg/kg dose of belantamab mafodotin on day 1 of every cycle for eight induction treatment cycles, wherein each induction treatment cycle is 21 days;
(ii) a 1.4 mg/kg dose of belantamab mafodotin on day 1 of every other cycle for eight induction treatment cycles, wherein each induction treatment cycle is 21 days;
(iii) a 1.9 mg/kg dose of belantamab mafodotin on day 1 of every other cycle for eight induction treatment cycles, wherein each induction treatment cycle is 21 days;
(iv) a 1.0 mg/kg dose of belantamab mafodotin on day 1 of every cycle for eight induction treatment cycles, wherein each induction treatment cycle is 21 days;
(v) a 1.4 mg/kg dose of belantamab mafodotin on day 1 of every cycle for eight induction treatment cycles, wherein each induction treatment cycle is 21 days;
(vi) a 1.4 mg/kg dose of belantamab mafodotin on day 1 of cycle 1 and a 1.0 mg/kg dose of belantamab mafodotin on day 1 of every third cycle from cycle 4 for eight induction treatment cycles, wherein each induction treatment cycle is 21 days;
(vii) a 1.9 mg/kg dose of belantamab mafodotin on day 1 of cycle 1 and a 1.4 mg/kg dose of belantamab mafodotin on day 1 of every third cycle from cycle 4 for eight induction treatment cycles, wherein each induction treatment cycle is 21 days;
(viii) a 1.9 mg/kg dose of belantamab mafodotin on day 1 of cycle 1 and cycle 4 and a 1.4 mg/kg dose of belantamab mafodotin on day 1 of every third cycle from cycle 7 for eight induction treatment cycles, wherein each induction treatment cycle is 21 days;
(ix) a 1.4 mg/kg dose of belantamab mafodotin on day 1 of cycle 1 and cycle 3 and a 1.0 mg/kg dose of belantamab mafodotin on day 1 of every third cycle from cycle 6 for eight induction treatment cycles, wherein each induction treatment cycle is 21 days; and
(x) a 1.0 mg/kg dose of belantamab mafodotin on day 1 of cycle 1 and cycle 5 for eight induction treatment cycles, wherein each induction treatment cycle is 21 days;
wherein the proteasome inhibitor is bortezomib, and wherein the bortezomib is administered to the subject at a dose of 1.3 mg/m² subcutaneously (SC) on days 1, 4, 8, and 11 of every 21-day cycle for eight induction treatment cycles;
wherein the immunomodulatory imide drug is lenalidomide, and wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and
wherein the corticosteroid is dexamethasone, and wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5.

22. The method of claim 21, further comprising administering to the subject, after the eight induction treatment cycles, a maintenance therapy comprising:
(a) a therapeutically effective dose of an anti-BCMA antigen binding protein;
(b) a therapeutically effective dose of an immunomodulatory imide drug; and
(c) a therapeutically effective amount of a corticosteroid,
wherein the anti-BCMA antigen binding protein is belantamab mafodotin, and wherein the belantamab mafodotin is administered intravenously according to a schedule selected from the group consisting of
(i) administration on day 1 of every 28-day cycle;
(ii) administration on day 1 of every other 28-day cycle; and
(iii) administration on day 1 of every third 28-day cycle;
wherein the immunomodulatory imide drug is lenalidomide, and wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and
wherein the corticosteroid is dexamethasone, and wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

23.-27. (canceled)

28. The method of claim 21, comprising administering to the subject:
belantamab mafodotin, wherein the belantamab mafodotin is administered intravenously to the subject at a dose of 1.4 mg/kg on day 1 of cycle 1 and at a dose of 1.0 mg/kg on day 1 of every third cycle from cycle 4 for eight induction treatment cycles, wherein each induction treatment cycle is 21 days;
bortezomib, wherein the bortezomib is administered to the subject at a dose of 1.3 mg/m² subcutaneously (SC) on days 1, 4, 8, and 11 of every 21-day cycle for eight induction treatment cycles;
lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and
dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and
administering to the subject, after the eight induction treatment cycles, a maintenance therapy comprising:
belantamab mafodotin, wherein the belantamab mafodotin is administered intravenously to the subject at a dose of 1.0 mg/kg on day 1 of every third cycle from cycle 9 onwards, wherein each cycle is 28 days;
lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and
dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

29. The method of claim 21, comprising administering to the subject:
belantamab mafodotin, wherein the belantamab mafodotin is administered intravenously to the subject at a dose of 1.9 mg/kg on day 1 of cycle 1 and at a dose of 1.4 mg/kg dose on day 1 of every third cycle from cycle 4 for eight induction treatment cycles, wherein each induction treatment cycle is 21 days;
bortezomib, wherein the bortezomib is administered to the subject at a dose of 1.3 mg/m² subcutaneously (SC) on days 1, 4, 8, and 11 of every 21-day cycle for eight induction treatment cycles;
lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and
dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and
administering to the subject, after the eight induction treatment cycles, a maintenance therapy comprising:
belantamab mafodotin, wherein the belantamab mafodotin is administered intravenously to the subject at a dose of 1.4 mg/kg on day 1 of every third cycle from cycle 9 onwards, wherein each cycle is 28 days;
lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and
dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

30. The method of claim 21, comprising administering to the subject:
belantamab mafodotin, wherein the belantamab mafodotin is administered intravenously to the subject at a dose of 1.9 mg/kg on day 1 of cycle 1 and cycle 4 and at a dose of 1.4 mg/kg dose on day 1 of every third cycle from cycle 7 for eight induction treatment cycles, wherein each induction treatment cycle is 21 days;
bortezomib, wherein the bortezomib is administered to the subject at a dose of 1.3 mg/m² subcutaneously (SC) on days 1, 4, 8, and 11 of every 21-day cycle for eight induction treatment cycles;
lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and
dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and
administering to the subject, after the eight induction treatment cycles, a maintenance therapy comprising:
belantamab mafodotin, wherein the belantamab mafodotin is administered intravenously to the subject at a dose of 1.4 mg/kg on day 1 of every third cycle from cycle 9 onwards, wherein each cycle is 28 days;
lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and
dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

31. The method of claim 21, comprising administering to the subject:
belantamab mafodotin, wherein the belantamab mafodotin is administered intravenously to the subject at a dose of 1.4 mg/kg on day 1 of cycle 1 and cycle 3 and at a dose of 1.0 mg/kg dose on day 1 of every third cycle from cycle 6 for eight induction treatment cycles, wherein each induction treatment cycle is 21 days;
bortezomib, wherein the bortezomib is administered to the subject at a dose of 1.3 mg/m² subcutaneously (SC) on days 1, 4, 8, and 11 of every 21-day cycle for eight induction treatment cycles;
lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and
dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and
administering to the subject, after the eight induction treatment cycles, a maintenance therapy comprising:
belantamab mafodotin, wherein the belantamab mafodotin is administered intravenously to the subject at a dose of 1.0 mg/kg on day 1 of every third cycle from cycle 9 onwards, wherein each cycle is 28 days;
lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and
dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

32. The method of claim 21, comprising administering to the subject:
belantamab mafodotin, wherein the belantamab mafodotin is administered intravenously to the subject at a dose of 1.0 mg/kg on day 1 of cycle 1 and cycle 5 for eight induction treatment cycles, wherein each induction treatment cycle is 21 days;
bortezomib, wherein the bortezomib is administered to the subject at a dose of 1.3 mg/m² subcutaneously (SC) on days 1, 4, 8, and 11 of every 21-day cycle for eight induction treatment cycles;
lenalidomide, wherein the lenalidomide is administered orally to the subject at a dose of (i) 25 mg on each of days 1-14 of each 21-day cycle for eight induction treatment cycles or (ii) 10 mg on each of days 1-14 of each 21-day cycle for eight induction treatment cycles if the subject has eGFR 30-60 mL/min/1.73 m²; and
dexamethasone, wherein the dexamethasone is administered to the subject orally at a dose of (i) 20 mg on days 1, 2, 4, 5, 8, 9, 11, and 12 of each 21-day cycle for eight induction treatment cycles or (ii) 10 mg on days 1, 2, 4, 5, 8, 9, 11, and 12 of each 21-day cycle for eight induction treatment cycles if the subject is older than 75 years or has a body mass index (BMI) of <18.5; and
administering to the subject, after the eight induction treatment cycles, a maintenance therapy comprising:
belantamab mafodotin, wherein the belantamab mafodotin is administered intravenously to the subject at a dose of 1.0 mg/kg on day 1 of every third cycle from cycle 9 onwards, wherein each cycle is 28 days;
lenalidomide, wherein the lenalidomide is administered orally to the subject at a dose of (i) 25 mg on each of days 1-21 of each 28-day cycle or (ii) 10 mg on days 1-21 of each 28-day cycle if the subject has eGFR 30-60 mL/min/1.73 m²; and
dexamethasone, wherein the dexamethasone is administered orally to the subject at a dose of (i) 40 mg on days 1, 8, 15 and 22 of each 28-day cycle or (ii) 20 mg on days 1, 8, 15, and 22 of each 28-day cycle if the subject is older than 75 years or has a body mass index (BMI) of <18.5.

33. The method of claim 21, wherein the multiple myeloma is newly diagnosed multiple myeloma.

34. The method of claim 33, wherein the subject is ineligible for autologous stem cell transplant.


SEQUENCE LISTING (10 sequences)

SEQ ID NO: 1 - protein, 5 aa, synthetic construct
NYWMH

SEQ ID NO: 2 - protein, 17 aa, synthetic construct
ATYRGHSDTYYNQKFKG

SEQ ID NO: 3 - protein, 12 aa, synthetic construct
GAIYDGYDVLDN

SEQ ID NO: 4 - protein, 11 aa, synthetic construct
SASQDISNYLN

SEQ ID NO: 5 - protein, 7 aa, synthetic construct
YTSNLHS

SEQ ID NO: 6 - protein, 9 aa, synthetic construct
QQYRKLPWT

SEQ ID NO: 7 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 8 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 9 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC